(19) United States
(12) Patent Application Publication
(10) Pub. No.: US 2019/0364935 A1
(43) Pub. Date: Dec. 5, 2019

(54) METHOD FOR INSPECTING LEGUME AND METHOD FOR PRODUCING LEGUME FOOD PRODUCT

(71) Applicant: NICHIREI FOODS INC., Tokyo (JP)

(72) Inventors: Shinya TSUKAMOTO, Chiba-shi (JP); Minoru MAMIYA, Chiba-shi (JP); Masahiko HONDA, Chiba-shi (JP)

(73) Assignee: NICHIREI FOODS INC., Tokyo (JP)

(21) Appl. No.: 16/477,761

(22) PCT Filed: Jan. 12, 2018
(86) PCT No.: PCT/JP2018/000687

§ 371 Date: Jul. 12, 2019

(30) Foreign Application Priority Data
Jan. 13, 2017 (JP) 2017-004722

(51) Int. Cl. A23L 5/20; G06T 7/00; A23L 11/00; A23L 5/10; A23L 3/26; G01N 23/083; G01N 23/18; G01N 33/02

(52) U.S. Cl. CPC A23L 5/20; G06T 7/0004; A23L 11/03; A23L 5/13; A23L 3/263; A23V 2002/00; G01N 23/18; G01N 33/025; G06T 2207/10116; G06T 2207/30128; G01N 23/083

(57) ABSTRACT

Provided is a legume inspecting method and a legume food manufacturing method for enabling a state of an inner part of a legume to be inspected with high accuracy without destroying the legume. The legume inspecting method includes a step of irradiating a legume which has not been wet-heated with X-rays, and acquiring X-ray image information from X-rays which have passed through the legume, and a step of inspecting an inner part of the legume based on the X-ray image information.

### TECHNICAL FIELD

The present invention relates to a legume inspecting method and a legume food manufacturing method, and particularly to a method for inspecting an inner part of a legume and a manufacturing method including the inspecting method.

### BACKGROUND ART

Legumes such as soybeans, peas, or broad beans including beans and pods covering the beans are in demand as fresh foods. Particularly, edamame, which are soybeans that have been harvested while they are immature and green, have also become widely popular as a frozen food which has been refrigerated after being cooked. As a legume inspecting method, destructive inspection for respectively extracting some of many legumes as samples and inspecting the samples has been known.

Patent Literature 1 discloses an inspecting method for turning on transmitted light from below a work table and transmitting the light to determine the presence or absence of intrusion of a foreign matter by visually observing a change in light amount and a change in color tone of the transmitted light.

Non Patent Literature 1 discloses that hair, insects, or the like can be detected by irradiating a food with near-infrared light having a higher food transmittance than that of visible light, imaging transmitted light using a special camera, and processing the image of the transmitted light.

### CITATION LIST

### Patent Literature

[Patent Literature 1] Japanese Patent Laid-Open No. 2006-162438

### Non Patent Literature

[Non Patent Literature 1] National University Corporation Toyohashi University of Technology Press Release, Apr. 24, 2014

### SUMMARY OF INVENTION

### Technical Problem

However, a conventional method for destroying and inspecting samples has problems that an omission of the inspection inevitably occurs so that inspection accuracy decreases because all foods cannot be inspected if the food is a food whose merchantability decreases when destroyed, for example, edamame.

The inspecting method in Patent Literature 1 is an inspecting method by visual observation using visible light, and thus, although a food need not be destroyed, has a problem that an inner part of the food is difficult to specifically inspect with visible light having a small transmittance.

The inspecting method in Non Patent Literature 1 has a risk that even near-infrared light does not reach an inner part of a food so that an inspection purpose cannot be accomplished. If a pod of a legume is thick, for example, near-infrared light which has impinged on the pod cannot reach its inner part by being diffused so that a state of the inner part of the pod is difficult to inspect.

The present invention is directed to providing a legume inspecting method and a legume food manufacturing method for enabling a state of an inner part of a legume to be inspected with high accuracy without destroying the legume.

### Solution to Problem

A legume inspecting method according to the present invention includes a step of inspecting a legume by irradiating the legume which has not been wet-heated with X-rays.

A legume food manufacturing method according to the present invention includes a step of inspecting a legume by irradiating the legume which has not been wet-heated with X-rays.

### Advantageous Effect of Invention

According to the present invention, by irradiating a legume with X-rays before the legume has not been wet-heated, a boundary between a pod and a bean is clear so that a bean area can be more reliably specified. Therefore, a state of an inner part of the legume can be inspected with high accuracy without destroying the legume.

### BRIEF DESCRIPTION OF DRAWINGS

FIG. 1 is a block diagram illustrating a configuration of an inspection apparatus according to the present embodiment;

FIG. 2 is a perspective view schematically illustrating a configuration of an X-ray imaging unit;

FIG. 3 is a flowchart illustrating a legume food manufacturing method according to the present embodiment;

FIG. 4 illustrates an X-ray image of a legume gotten by an inspecting method according to the present embodiment;

FIG. 5 illustrates an X-ray image of a legume after wet heating, where FIG. 5A is an image after boiling, and FIG. 5B illustrates an image after braising;

FIG. 6 is a list illustrating a relationship between a tube voltage and an X-ray image in the X-ray imaging unit;

FIG. 7 is a diagram for describing a non-defective product model, where FIG. 7A illustrates an X-ray image of a legume, FIG. 7B is a graph illustrating an X-ray transmission intensity, and FIG. 7C illustrates a binary data image;

FIG. 8 is a diagram for describing a defective product model, where FIG. 8A illustrates an X-ray image of a legume, FIG. 8B is a graph illustrating an X-ray transmission intensity, and FIG. 8C illustrates a binary data image;

FIG. 9 is a schematic view for describing a non-defective product model;

FIG. 10 is a diagram for describing a legume which is a non-defective product, where FIG. 10A illustrates a non-defective product model, and FIG. 10B illustrates an X-ray image of the non-defective product;

FIG. 11 is a diagram for describing a legume which is a defective product (1), where FIG. 11A illustrates a defective product model, and FIG. 11B illustrates an X-ray image of the defective product;

FIG. 12 is a diagram for describing a legume which is a defective product (2), where FIG. 12A illustrates a defective product model, and FIG. 12B illustrates an X-ray image of the defective product;

FIG. 13 is a diagram for describing a legume which is a defective product (3), where FIG. 13A illustrates a defective product (depression) model, FIG. 13B illustrates an X-ray image of a defective product (depression), FIG. 13C illustrates a defective product (discoloration) model, and FIG. 13D illustrates an X-ray image of a defective product (discoloration);

FIG. 14 is a diagram for describing a legume which is a defective product (4), where FIG. 14A illustrates a defective product model, and FIG. 14B illustrates an X-ray image of the defective product; and

FIG. 15 illustrates an X-ray image of a legume which has been heated with dry heat, gotten by the inspecting method according to the present embodiment.

### DESCRIPTION OF EMBODIMENT

An embodiment of the present invention will be described in detail below with reference to the drawings.

### Entire Configuration

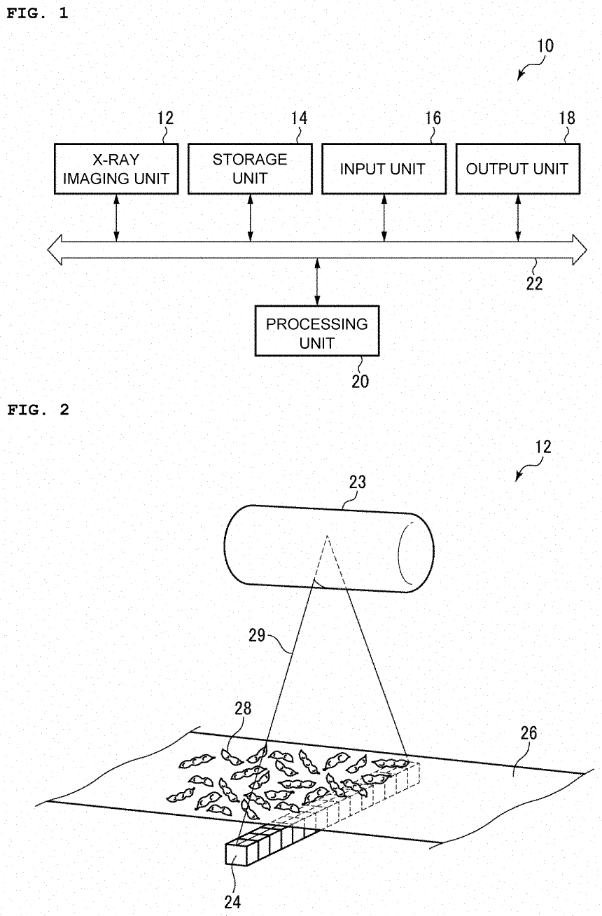

An inspection apparatus 10 illustrated in FIG. 1 includes an X-ray imaging unit 12, a storage unit 14, an input unit 16, an output unit 18, and a processing unit 20, which are connected to one another via a bus 22. The processing unit 20 reads out application programs such as a basic program and an image processing program previously stored, and controls the entire inspection apparatus 10 according to the various programs. The processing unit 20 can execute a plurality of programs (application programs, etc.) in parallel.

The storage unit 14 includes at least one of a semiconductor memory device, a magnetic tape device, a magnetic disk device, and an optical disk device, for example. The storage unit 14 stores an operating system program, a driver program, an application program, data, and the like used for processing in the processing unit 20. For example, the storage unit 14 stores an inspection program for causing the processing unit 20 to perform inspection processing for inspecting an inner part of a legume, for example, as the application program. The inspection program may be installed into the storage unit 14 using a known setup program or the like from a computer readable portable recording medium such as a CD-ROM (compact disc read-only memory) or a DVD-ROM (digital versatile disc read-only memory).

The storage unit 14 stores non-defective product data of a bean which is a non-defective product and an X-ray image, described below. The non-defective product data is bean information which the bean which is the non-defective product should have, e.g., information obtained by quantifying its size (area) and shape. Further, the storage unit 14 may temporarily store temporary data relating to predetermined processing.

The input unit 16 may be any device if it can input data, and is a touch panel or a keyboard, for example. An operator can input characters, numerals, signs, and the like using the input unit 16. The input unit 16 generates, when operated by the operator, a signal corresponding to the operation. The generated signal is fed to the processing unit 20 as an instruction issued by the operator.

The output unit 18 may be any device if it can display a video, an image, and the like, and is a liquid crystal display or an organic EL (electro-luminescence) display, for example. The output unit 18 displays an image corresponding to image data, for example, inputted from the processing unit 20. Also, the output unit 18 may be a device which prints images or characters on a display medium such as paper.

As illustrated in FIG. 2, the X-ray imaging unit 12 irradiates a legume 28 with X-rays 29 and receives X-rays which have passed through the legume 28, to acquire X-ray image information. The X-ray imaging unit 12 includes an X-ray irradiator 23, an X-ray receiver 24, and a belt conveyor 26 serving as a conveyance unit. The X-ray irradiator 23 and the X-ray receiver 24 are arranged to oppose each other in a vertical direction with the belt conveyor 26 sandwiched therebetween. The belt conveyor 26 conveys a plurality of legumes 28 in one direction. The plurality of legumes 28 on the belt conveyor 26 are arranged with the legumes 28 not overlapping one another in the vertical direction.

The X-ray irradiator 23 irradiates the legumes 28 to be conveyed on the belt conveyor 26 with the X-rays 29 from above. The X-ray irradiator 23 preferably irradiates the X-rays under an irradiation condition that a tube voltage is 25 kV to 50 kV. If the tube voltage in the X-ray irradiator 23 is less than 25 kV, a gradation of the inner part of the legume becomes unclear in the X-ray image. If the tube voltage in the X-ray irradiator 23 exceeds 50 kV, a difference in tint between the bean and a pod is small in the X-ray image so that an outer shape of the bean becomes difficult to specify.

The X-ray receiver 24 is a line sensor the length in a longitudinal direction of which is substantially the same as the width of the belt conveyor 26. The X-ray receiver 24 receives X-rays which have passed through the legume 28 and outputs obtained X-ray image information to the processing unit 20 via a LAN (local area network), which is not illustrated, and the bus 22.

### Legume Food Manufacturing Method

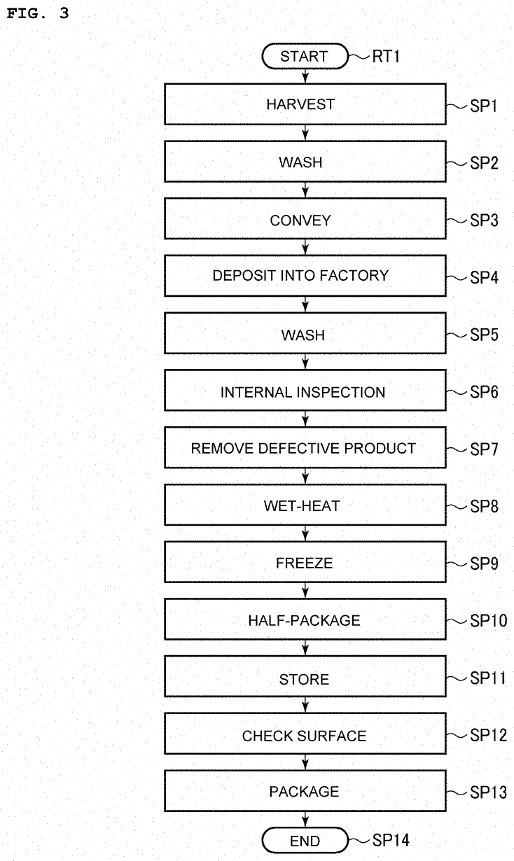

Then, a method for manufacturing legume foods from the legumes 28 harvested in a field will be described with reference to FIG. 3. After the legumes 28 are first harvested in step SP1, the legumes 28 are simply washed (step SP2). The washed legumes 28 are conveyed from the field (step SP3), and are deposited into a factory (step SP4). In the factory, the legumes 28 are washed again a plurality of times (step SP5). Internal inspection is performed (step SP6) for the legumes 28 which have been washed.

In the internal inspection (step SP6), the X-ray imaging unit 12 acquires respective X-ray image information about the legumes 28 which are being conveyed. The processing unit 20 reads out the X-ray image information. The processing unit 20 respectively extracts features of the legumes 28 from the X-ray image information, to obtain feature information. The processing unit 20 determines whether the legumes 28 are each non-defective or defective based on the feature information.

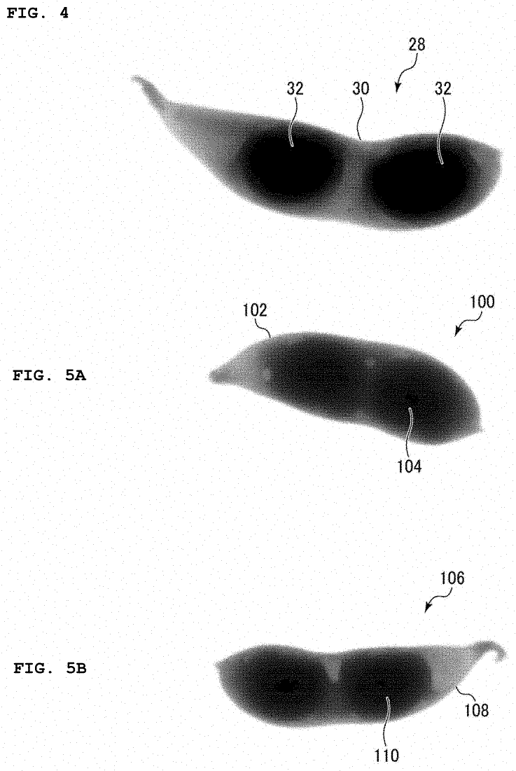

FIG. 4 illustrates an X-ray image of the legume 28 generated from the X-ray image information obtained in the above described procedure. A bean 32 inside a pod 30 is reflected on the X-ray image. The processing unit 20 specifies an area of the bean 32 from the X-ray image information, for example. An area of the bean in the X-ray image is displayed to be blacker because it does not more easily transmit X-rays than an area of the pod 30. The processing unit 20 specifies an area displayed to be black as the bean area.

Although not illustrated in the drawing, the processing unit 20 specifies a definite area outside the bean area in a gradation which differs from those of the bean area and the pod area as a foreign matter other than the bean 32, e.g., an insect. Further, the processing unit 20 specifies that there is an external form abnormality (deformation, discoloration, or vermiculation) on a surface of the bean 32 when there is partially a dark shaded site or a light shaded site within the bean area. In the above described manner, the processing unit 20 obtains feature information about the legume 28.

Then, the processing unit 20 determines whether the legume 28 is non-defective or defective based on the feature information obtained in the above described manner. For example, the processing unit 20 compares the bean area with the non-defective product data. As a result of the comparison, the processing unit 20 determines the bean area to be a non-defective product when a difference between the bean area and the non-defective product data is a predetermined value or less. On the other hand, the processing unit 20 determines the bean area to be a defective product when the difference between the bean area and the non-defective product data exceeds the predetermined value.

For example, the processing unit 20 reads out image data of the bean which is the non-defective product as the non-defective product data from the storage unit 14, and compares the image data with the bean area. The processing unit 20 calculates a mismatch rate of the bean area with the non-defective product data. A threshold value of the mismatch rate for separating a non-defective product and a defective product is previously determined from the legume 28 which is a non-defective product. If the mismatch rate is the predetermined threshold value or less, the legume 28 is determined to be the non-defective product. If the mismatch rate exceeds the threshold value, the legume 28 is determined to be the defective product. The processing unit 20 can determine whether the size of the bean 32 is non-defective or defective and whether there is an external form abnormality by comparing the bean area with the non-defective product data. Also, the processing unit 20 determines the legume 28 to be the defective product when it detects an insect within the legume 28.

Then, the legumes 28 which have been determined to be defective products are removed from the belt conveyor 26 (step SP7). Then, the legumes 28 which are determined to be non-defective products are wet-heated (step SP8). Here, the wet heating means a cooking operation for performing heating using water as a heat medium, and specifically means braising, boiling, and an operation for pouring hot water (e.g., water having a temperature of 80° C. or more).

The legumes 28 which have been wet-heated are frozen (step SP9), are then temporarily packaged (half-packaged) (step SP10), and are stored for a predetermined period (step SP11). Finally, surfaces of the legumes 28 are checked (step SP12), and the legumes 28 are repackaged into small sizes (step SP13) and are then respectively shipped as the legume foods.

### Operation and Effect

The X-ray imaging unit 12 acquires the X-ray image information obtained by irradiating the X-rays 29 from above onto the legumes 28 which are being conveyed, and outputs the acquired X-ray image information to the processing unit 20. The processing unit 20 acquires the feature information for each of the legumes 28 based on the X-ray image information inputted from the X-ray imaging unit 12, and compares the feature information with the non-defective product data, to determine whether the legume 28 is non-defective or defective. The inspection apparatus 10 may input a determination result to the output unit 18. In this case, the output unit 18 can display an output result corresponding to the determination result, together with the X-ray image generated from the X-ray image information.

In the present embodiment, the legume 28 which has not been wet-heated is irradiated with the X-rays 29, to obtain the X-ray image information about the legume 28. Thus, a boundary between the pod 30 and the bean 32 is clear so that the bean area can be more reliably specified, as illustrated in FIG. 4. On the other hand, when the legume 28 is wet-heated, water penetrates into the pod 30 so that the bean area becomes difficult to specify. In a legume 100 which has been boiled (at 100° C. and for three minutes) as the wet heating, water penetrates into a pod 102 so that a boundary between a bean 104 and the pod 102 becomes unclear, as illustrated in FIG. 5A. The weight of the legume 28 after the boiling is 102% of that before the boiling. Also in a legume 106 (FIG. 5B) which has been braised (at 100° C. and for ten minutes) as the wet heating, water has penetrated into a pod 108 so that a boundary between a bean 110 and the pod 108 is unclear so that a bean area is difficult to specify. The weight of the legume 28 after the braising is 102% of that before the braising.

In an environment where a sufficient amount of water which penetrates into a pod exists around a legume, when the legume is heated to a temperature at which a tissue of the legume is softened, the water penetrates into the pod. Here, the sufficient amount of water means an amount of water which remains as a liquid after penetrating into the pod. Therefore, heating which does not satisfy the above described condition is not included in wet heating. Washing and heating with warm water having such a temperature that water does not penetrate into the pod, for example, a temperature of less than 80° C. are not included in the wet heating in this specification. Heating not using water as a heat medium, for example, hot-air heating or microwave heating is not included in the wet heating.

In an inspecting method according to the present embodiment, it is determined whether the legume 28 which has not been wet-heated is non-defective or defective based on the X-ray image information about the legume 28 obtained by irradiating the legume 28 with the X-rays 29. Thus, a state of the inner part of the legume 28 can be inspected with high accuracy without the legume 28 being destroyed.

In the inspecting method according to the present embodiment, the respective X-ray image information about all the legumes 28 to be conveyed on the belt conveyor 26 can be obtained so that all the legumes 28 can be inspected. Therefore, the legume food manufacturing method includes the inspecting method so that legume foods each not including a defective product can be easily manufactured.

In the inspecting method according to the present embodiment, the legumes 28 which have been determined to be non-defective products depending on the size and the shape of the bean 32, for example, can also be further graded based on the X-ray image information about all the legumes 28.

Then, a result of examination of a relationship between a tube voltage in the X-ray irradiator 23 and an X-ray image will be described. The tube current was set to 2 mA. A non-defective product was evaluated, based on obtained X-ray image information, in a procedure for cutting out an image (1) of a legume from an entire image, cutting out an image (2) of a bean from the image (1), and determining an irregular shape of the bean from a gradation of the image (2).

An evaluation point of a non-defective product was 5 when a contour is clear and a difference in tint between a pod and a bean is large, 4 when the contour is clear but the pod is reflected to be black so that a difference in tint between the pod and the bean is small, 3 when the contour becomes slightly blurred, 2 when the contour is not very clear, and 1 when the contour is not reflected. An evaluation point of a defective product was 5 when a gradation of an inner part of a legume is very clear and there is a difference in tint between a bean and a pod, 4 when the gradation of the inner part of the legume is clear and there is a difference in tint between the bean and the pod, 3 when the gradation of the inner part of the legume is clear but a difference in tint between the bean and the pod is small, 2 when the gradation of the inner part of the legume is not clear and respective gradations of the bean and the pod are large, and 1 when the gradation of the inner part of the legume is not clear and the respective gradation s of the bean and the pod are small. In actual determination, features based on a foreign matter (an insect, etc.) in the pod outside a bean area and a size and a shape of the bean itself are acquired from a contour of the bean. Then, features based on a foreign matter (an insect, etc.) existing in the bean area and discoloration of the bean are acquired from a gradation of the bean area. Comprehensive determination was set as C by judging that the determination is significantly difficult if the evaluation point of the non-defective product or the defective product is 2 or less, C by judging that the determination is difficult from the contour of the bean or the feature in the bean area if a total of the respective evaluation points of the non-defective product and the defective product is 6 or less, B by judging that the determination can be performed because at least one of the non-defective product and the defective product can be favorably determined if the total is 7 or more, and A by judging that the determination can be more reliably performed because both the non-defective product and the defective product can be reliably determined if the total is 10.

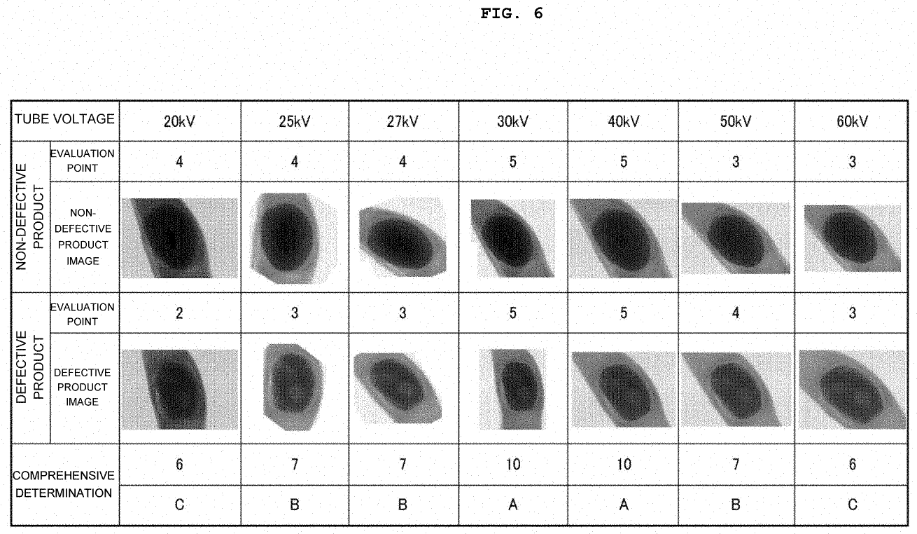

The result is given in a list illustrated in FIG. 6. It can be confirmed that the non-defective product and the defective product can be determined when the tube voltage is in a range of 25 kV to 50 kV and a clearer X-ray image is more preferably obtained when the tube voltage is in a range of 30 kV to 40 kV.

A method for determining whether a legume is non-defective or defective from feature information is not particularly limited. For example, contour data, binary data, and gradation data may be acquired as the feature information. The contour data is a contour line of a bean extracted from X-ray image information. The processing unit recognizes a portion which is greatly different from a contour line of a non-defective product to be defective, to thereby detect chipping or depression due to vermiculation, an insect adjacent to a bean, and a bean having a small grain diameter (a defective size of the bean).

The binary data is data simplified by binarizing the vicinity of a bean area in X-ray image information. The processing unit detects a defect such as discoloration or depression from the simple binary data. The gradation data is data not merely binarized but having integration information about a gradation. The processing unit detects a defect such as discoloration or depression by indexes such as the magnitudes of changes in concentration and brightness.

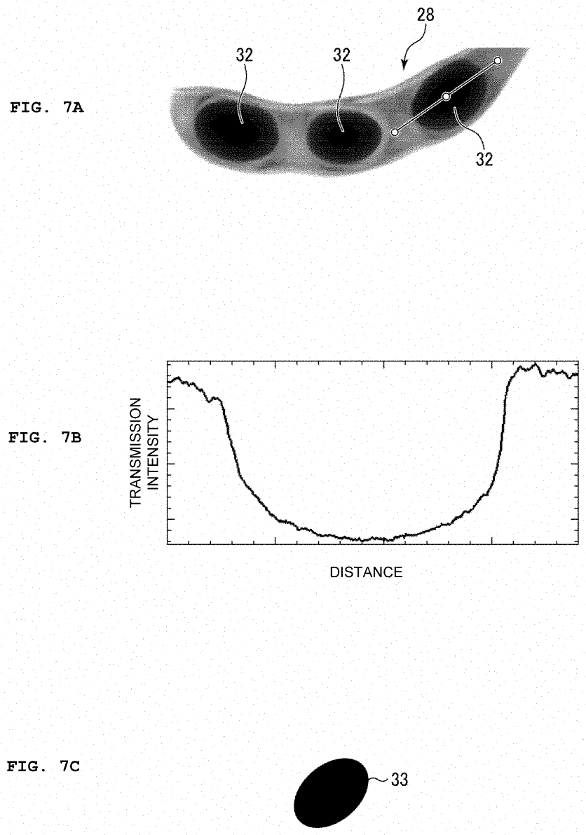

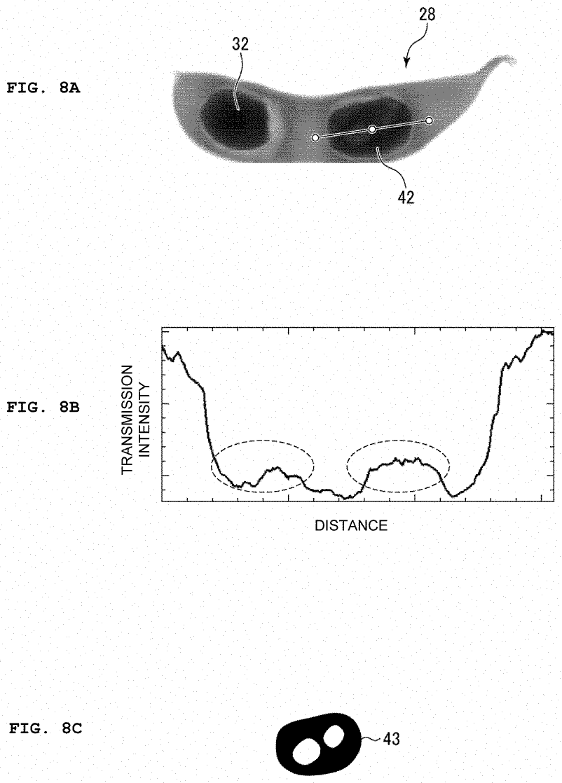

A specific example of a case where a defect that is discoloration is detected based on binary data will be described with reference to FIG. 7 and FIG. 8. The processing unit acquires binary data obtained by binarizing the vicinity of a bean area based on X-ray image information illustrated in FIG. 7A (FIG. 7C). The binary data is compared with non-defective product data. In the drawing, discoloration is not observed in the binary data of the bean area so that a mismatch rate is a threshold value or less. Therefore, the processing unit determines the bean to be a non-defective product. FIG. 7B illustrates an X-ray transmission intensity of a straight line portion in FIG. 7A. In FIG. 7B, a horizontal axis represents a distance, and a vertical axis represents an X-ray transmission intensity. FIG. 7B shows that an anomalous peak is not observed in the bean area so that there is neither discoloration nor depression in the bean 32.

On the other hand, FIG. 8A illustrates X-ray image information about a bean which is discolored and deformed. When the vicinity of a bean area is binarized based on the X-ray image information illustrated in FIG. 8A, a discolored portion becomes apparent (FIG. 8C). In a case illustrated in the drawing, when binary data of the bean area and non-defective product data are compared with each other, a mismatch rate exceeds a threshold value. Therefore, the processing unit determines the bean to be a defective product. FIG. 8B illustrates an X-ray transmission intensity of a straight line portion in FIG. 8A. In FIG. 8B, a horizontal axis represents a distance, and a vertical axis represents an X-ray transmission intensity. FIG. 8B shows that two peaks are observed in the bean area so that the bean 42 is defective, for example, discolored.

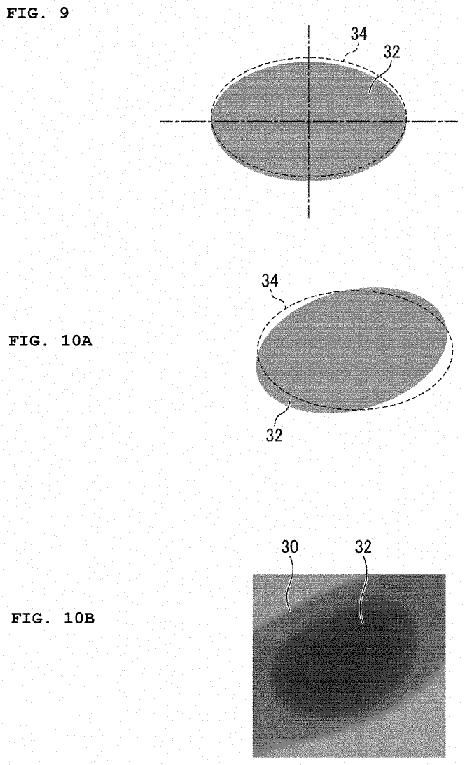

Then, in the inspecting method according to the present embodiment, specific examples of legumes 28 which can be determined to be non-defective products and defective products will be described below. Note that in the following description, non-defective product data 34 means image data of a bean which is a non-defective product. The legume 28 is determined to be a non-defective product if a mismatch rate between a bean area 32 specified by the processing unit 20 and the non-defective product data 34 is a threshold value or less, as illustrated in FIG. 9.

FIG. 10A and FIG. 10B to FIG. 14A and FIG. 14B each illustrate an example in which an X-ray image generated based on X-ray image information obtained by irradiating an edamame serving as a legume 28 with X-rays having a tube voltage of 30 kV and having a tube current of 2 mA, feature information obtained from the image, and non-defective product data are compared with one another. The edamame illustrated in FIG. 10A and 10B are each determined to be a non-defective product because a mismatch rate between non-defective product data 34 and a bean area 32 is a threshold value or less.

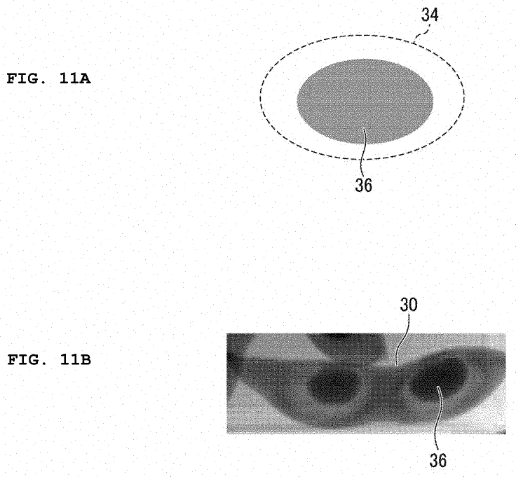

FIG. 11A and FIG. 11B each illustrate an example of a defective product which is a bean 36 having a small grain diameter. The edamame illustrated in the drawings is determined to be a defective product because a mismatch rate between non-defective product data 34 and the bean area 36 exceeds a threshold value.

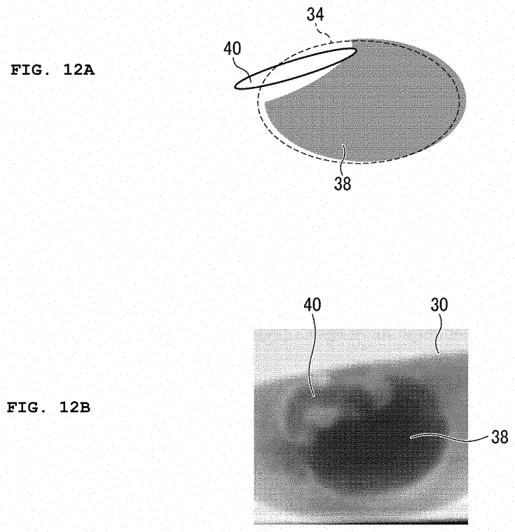

FIG. 12A and FIG. 12B each illustrate an example of a defective product in which an insect 40 exists inside a pod 30 and a bean 38 is chipped. An edamame in the drawings is determined to be a defective product because the bean 38 is smaller than non-defective product data 34 by an amount of chipping due to vermiculation and a mismatch rate exceeds a threshold value. Further, the processing unit 20 specifies a definite area in a gradation different from those of a bean area and an area of the pod 30 in a site adjacent to the bean 38 as the insect 40, and determines the edamame to be a defective product from this viewpoint.

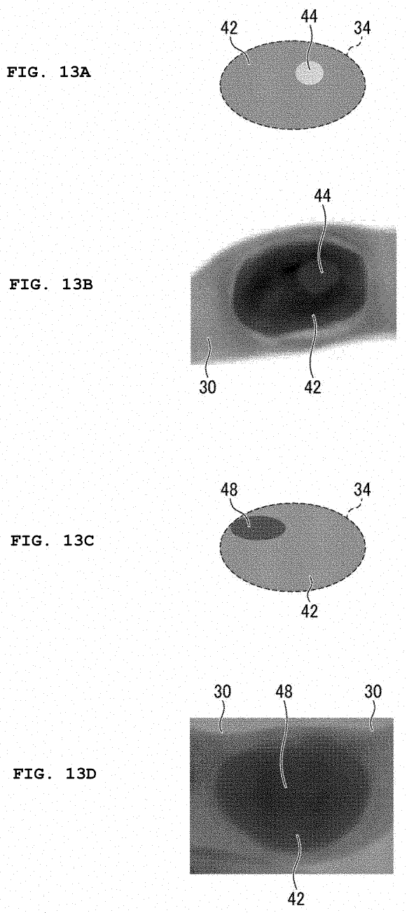

FIG. 13A to FIG. 13D each illustrate an example of a defective product having an external form abnormality (deformation or discoloration) on a surface of a bean 42 (FIG. 13A and FIG. 13C). When the bean 42 has its surface depressed, a gradation in a corresponding portion becomes light in an X-ray image (FIG. 13B). On the other hand, when the bean 42 has its surface discolored, a gradation in a corresponding portion becomes dark in the X-ray image (FIG. 13D). The processing unit 20 can determine the bean 42 to be a non-defective product because a mismatch rate between the bean area 42 and non-defective product data 34 is a threshold value or less from a viewpoint of size. However, the processing unit 20 specifies the bean area 42 as having an external form abnormality and determines the edamame to be a defective product because there is partially a light-shaded site 44 or a dark-shaded site 48 within the bean area 42 so that a mismatch rate exceeds a threshold value from a viewpoint of color.

FIG. 14A and FIG. 14B each illustrate an example of a defective product in which an insect 40 exists adjacent to a bean 32 (FIG. 14A). The processing unit 20 determines the bean itself to be a non-defective product because a mismatch rate between the bean area 32 and non-defective product data 34 is a threshold value or less. However, the processing unit 20 specifies a definite area in a different gradation from those of the bean area 32 and an area of a pod 30 as the insect 40 (FIG. 14B), and determines the edamame to be a defective product from this viewpoint.

### Modification

The present invention is not limited to the above described embodiment, but can be appropriately changed without departing from the scope and spirit of the present invention.

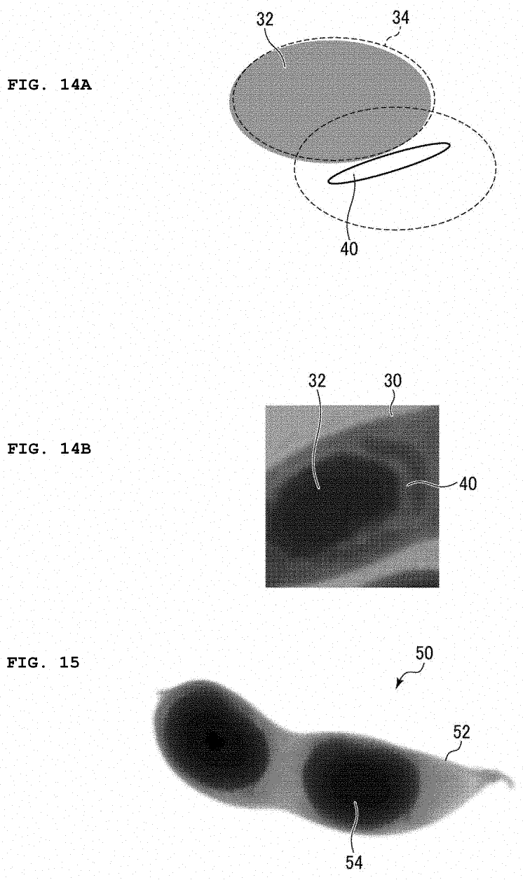

Although a case where the legume 28 which has not been wet-heated is inspected has been described in the above described embodiment, the legume 28 may be inspected, when heating does not use water as a heat medium, after the heating. FIG. 15 illustrates an X-ray image gotten by irradiating a legume 50 heated with dry heat (at 160° C. and for eight minutes) with X rays having a tube voltage of 30 kV and having a tube current of 2 mA. FIG. 15 shows that a boundary between a pod 52 and a bean 54 is clear because water does not penetrate into the pod 52 so that the bean area 54 can be more reliably specified. The weight of the legume 50 after the heating with dry heat was 61% of that before the heating.

Although a case where internal inspection is performed immediately before wet heating (step SP8) has been described in the above described embodiment, the present invention is not limited to this. For the legume 28 which has not been wet-heated, internal inspection may be performed at a timing at which the legume 28 has not been heated at all, e.g., at a timing after washing (step SP2) after harvesting and before conveyance (step SP3) and a timing after deposition into a factory (step SP4) and before washing (step SP5).

Although a case where one condition of the tube voltage is determined to inspect the inner part of the legume has been described in the above described embodiment, the present invention is not limited to this. For example, the inner part of the legume may be inspected based on a plurality of X-ray image information acquired by X-rays irradiated under a plurality of conditions. More specifically, it may be inspected whether each legume is non-defective or defective by respectively selecting images under conditions suited to inspect a pod, an outer shape of a bean, and a surface of the bean from a plurality of X-ray image information which differ in the condition of the tube voltage.

### REFERENCE SIGNS LIST

28 Legume

29 X-rays

30 Pod

32 Bean

40 Insect

1. A legume inspecting method, the method comprising a step of inspecting a legume by irradiating the legume which has not been wet-heated with X-rays.

2. The legume inspecting method according to claim 1, wherein an inner part of the legume is inspected based on X-ray image information acquired from X-rays which have passed through the legume.

3. The legume inspecting method according to claim 1, wherein an irradiation condition of the X-rays is that a tube voltage is not less than 25 kV nor more than 50 kV.

4. The legume inspecting method according to claim 1, wherein the legume is an edamame.

5. The legume inspecting method according to claim 1, wherein the inspecting step includes detecting a foreign matter inside a pod of the legume, a defective shape of a bean in the legume, a defective size of the bean, or discoloration of the bean.

6. The legume inspecting method according to claim 1, wherein the wet heating is braising, boiling, or an operation for pouring hot water.

7. A legume food manufacturing method, the method comprising a step of inspecting a legume by irradiating the legume which has not been wet-heated with X-rays.

8. The legume food manufacturing method according to claim 7, the method further comprising
a step of wet-heating a legume which has been determined to be a non-defective product in the inspection,
wherein the inspecting step includes inspecting an inner part of the legume based on X-ray image information acquired from X-rays which have passed through the legume.

9. The legume food manufacturing method according to claim 7, wherein an irradiation condition of the X-rays is that a tube voltage is not less than 25 kV nor more than 50 kV.

10. The legume food manufacturing method according to claim 7, wherein the legume is an edamame.

11. The legume food manufacturing method according to claim 7, wherein the inspecting step includes detecting a foreign matter inside a pod, a defective shape of a bean, a defective size of the bean, or discoloration of the bean.

12. The legume food manufacturing method according to claim 7, wherein the wet heating is braising, boiling, or an operation for pouring hot water.

13. The legume inspecting method according to claim 1, wherein the inspecting step includes inspecting a defect which is detectable based on a contour of a bean and a defect which is detectable based on a shade of a bean area.

14. The legume food manufacturing method according to claim 7, wherein the inspecting step includes inspecting a defect which is detectable based on a contour of a bean and a defect which is detectable based on a shade of a bean area.
